(19) United States
(12) Patent Application Publication
(10) Pub. No.: US 2015/0025008 A1
(43) Pub. Date: Jan. 22, 2015

(54) Compositions and Methods of Detecting and Treating Neural Tube Defects

(71) Applicant: Ann and Robert H. Lurie Children's Hospital of Chicago, Chicago, IL (US)

(72) Inventors: Chandra Shekhar Mayanil, Chicago, IL (US); Elise Allender, Chicago, IL (US); Takao Tsurubuchi, Tsukuba (JP); Norman Ginsberg, Chicago, IL (US); David G. McLone, Chicago, IL (US); Tadanori Tomita, Chicago, IL (US)

(21) Appl. No.: 14/331,410

(22) Filed: Jul. 15, 2014

Related U.S. Application Data
(60) Provisional application No. 61/847,662, filed on Jul. 18, 2013.

(51) Int. Cl. G01N 33/68; G01N 33/74; A61K 31/4418; A61K 38/18; A61K 31/519

(52) U.S. Cl. CPC G01N 33/6893; A61K 38/18; A61K 31/519; A61K 31/4418; G01N 33/74; G01N 2333/51; G01N 2333/47; G01N 2333/475; G01N 2800/385; G01N 2800/52
USPC 514/8.3; 514/252.16; 514/357; 435/7.1; 435/7.4; 435/7.92

(57) ABSTRACT

The present invention generally relates to compositions, reagents and methods for detecting and treating a neural tube defect in a fetus. One aspect of the invention provides a method including administrating a composition containing noggin or LDN-193189 to the fetus in utero. In certain embodiments, the composition is administrated if the maternal blood or amniotic fluid contains an elevated amount of BMP4 and/or a reduced amount of noggin. In another aspect of the invention, the method includes administrating a composition containing GDC-0449 to the fetus in utero. In certain embodiments, the GDC-0449 is administrated if the maternal blood or amniotic fluid contains an elevated amount of sonic hedgehog.

### RELATED APPLICATIONS

This patent application claims the benefit of U.S. provisional patent application No. 61/847,662, filed Jul. 18, 2013, the entire contents of which application is hereby incorporated by reference.

### TECHNICAL FIELD

The present invention generally relates to compositions and methods for detecting and treating neural tube defects.

### BACKGROUND

Neural tube defects (NTDs), such as myelomeningocele (MM), anencephaly, holoprosencephaly, and encephalocele are relatively common, with a collective prevalence of about 1/1000 pregnancies. In the United States over 90% of infants born with spina bifida survive the first year of life with varying levels of sensory loss and paralysis. At least 75% of children born with a MM can be expected to reach their early adult years, however late deterioration is common. When NTDs are detected, it is vital to provide the future parent(s) with as much information as possible, so that informed decisions can be made for both the mother and child. NTDs can be difficult to predict, and prenatal screening and diagnosis can be variable. Although second-trimester sonographic diagnosis of NTDs is accurate and detection of aneuploidies and NTDs at 11-13 weeks of gestation is improving, there continues to be a need for reliable, early detection, with greater prediction of outcome, and ultimately development of innovative pharmacological treatment for in utero closure.

Early detection with prediction of NTD severity would allow patients in both developed, and developing countries, where testing for NTDs is becoming more prevalent, to make informed decisions about a healthy outcome for the fetus and mother. Early detection would be especially beneficial for those individuals with a high possibility of NTD associated pregnancy. There is recurrent risk of NTD following miscarriage, and a history of previous miscarriages is a risk indicator for anencephaly in future gestations. Recent studies on the etiology of human NTDs, as well as analysis of mouse models, suggest the involvement of epigenetic factors in determining susceptibility.

Existing treatment of neural tube defects relies on surgical intervention and stitching back the open neural tube immediately after birth. Morbidity is high and those that survive are wheelchair bound, and have no bladder control. Although maternal folic acid supplementation prior to conception and during neural tube development rescues neural tube defects, this treatment is effective only in approximately 60-70% of human cases. A certain percentage of NTDs are non-responsive to folic acid.

Even in cases of folate responsive NTDs, these are times folic acid supplementation advice might be inadvertently neglected by a woman of child bearing age. By the time a woman realizes that she is pregnant, disruption of neural tube development has already taken place and by the time a pregnant woman comes to know that the fetus has MM it is too late for clinical intervention other than performing in utero surgery. In utero surgery however, has its own drawbacks and is not bereft of maternal and fetal morbidity. Those that survive are wheelchair bound, and have no bladder control.

### BRIEF SUMMARY

One aspect of the present invention provides a method of treating a NTD in a fetus. The method can include administrating a composition including noggin or LDN-193189 in utero. In one embodiment, the composition is administrated if the maternal blood or amniotic fluid contains a reduced amount of noggin and/or an elevated amount of BMP4. In other embodiments, the composition is administrated if an amniotic fluid stem cell exhibits an elevated level of at least one of H3K4 methylation and H3K27 methylation or a decreased level of at least one of H3K9 acetylation, H3K18 acetylation and Gcn5. In yet other embodiments, the composition is administrated if the maternal blood or amniotic fluid contains an elevated amount of BMP4 and an amniotic fluid stem cell exhibits an elevated level of at least one of H3K4 methylation and H3K27 methylation or a decreased level of at least one of H3K9 acetylation, H3K18 acetylation and Gcn5.

Another aspect of the present invention provides a method of treating a neural tube defect in a fetus including administrating a composition including GDC-0449 in utero. In one embodiment, the composition including GDC-0449 is administrated if the maternal blood or amniotic fluid contains an elevated amount of sonic hedgehog (Shh). In other embodiments, the composition including GDC-0449 is administrated if an amniotic fluid stem cell exhibits an elevated level of at least one of H3K9 acetylation, H3K18 acetylation and Gcn5. In yet other embodiments, the composition including GDC-0449 is administrated if the maternal blood or amniotic fluid contains an elevated amount of sonic hedgehog (Shh) and an amniotic fluid stem cell exhibits an elevated level of at least one of H3K9 acetylation, H3K18 acetylation and Gcn5.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows photographs illustrating the expression of stem cell markers by amniotic fluid stem cells. Neurospheres were grown from total AFSCs. Maternal cells do not survive in neurosphere culture medium (Neurobasal plus bFGF and EGF). Embryonic stem cells grow in Neurobasal plus bFGF and EGF and form neurospheres colonies, these cells are referred to as Amniotic Fluid Stem Cells (AFSCs). Colonies were immunostained for Sox2, Oct4, CD133 and nestin. DAPI was used to stain nuclei. Sox2, Oct4 and CD 133 and nestin were present in AFSC from (A) normal (A) MM (B) and anencephaly (C) affected pregnancies. Each experiment was done at least 4 times and in triplicate.

FIG. 2 shows photographs illustrating that amniotic fluid stem cells when differentiated express different differentiation markers. Neurospheres were grown in Neurobasal medium minus growth factors for 7 days and subsequently immuno-stained for astrocyte (GFAP), neuron (TuJ1) and oligodendrocytes (O4) markers. DAPI was used to stain nuclei. GFAP and TuJ1 were at similar levels in AFSC obtained from normal (A), MM (B) and Anencephaly (C) affected pregnancies. O4 staining was: MM>Anencephaly>normal. Each experiment was done at least 4 times and in triplicate.

FIG. 3 shows photographs illustrating immunostaining (H3K4) of cultured AFSCs from normal and MM and anencephaly affected pregnancies: (A) H3K4me2 and Oct 4; (B) H3K4me3 and Oct4; all cells were counterstained with DAPI nuclear stain. Presence of Oct4, a stem cell marker, demonstrates that stained cells are of embryonic, not maternal origin. Both H3K4me2 and H3K4me3 immunostaining increased with MM but not anencephaly. Each experiment was done at least 4 times and in triplicate.

FIG. 4 shows photographs illustrating immunostaining (H3K27 methylation and KDM6B) of cultured AFSCs from normal and MM and anencephaly affected pregnancies: (A) H3K27me2 and Oct 4; (B) H3K27me3 and Oct4; (C) KDM6B and Oct4; all cells were counterstained with DAPI nuclear stain. H3K27me2 and H3K4me3 immunostaining increased with MM, only H3K27me3 increased with anencephaly. KDM6B immunostaining decreased with both MM and anencephaly. Each experiment was done at least 4 times and in triplicate.

FIG. 5 shows photographs illustrating immunostaining (H3K9 and H3K18 acetylation and Gcn5) of AFSCs from normal and MM and anencephaly affected pregnancies: (A) H3K9Ac and Gcn5; (B) H3K18Ac and Gcn5; all cells were counterstained with DAPI nuclear stain. Both H3K4me2 and H3K4me3 immunostaining increased with MM but not anencephaly. Each experiment was done at least 4 times and in triplicate.

FIG. 6 is a graph showing results for epigenetic markers. A minimum of 6 different fields of immunostained cells were measured using densitometry and an overall value represented as an arbitrary densitometry unit with Oct4=1, is shown. Compared to normal, the staining intensity for H3K4me2, H3K4me3, H3K27me2 and H3K27me3 was high in AFSCs derived from MM affected pregnancy. Staining intensity for H3K27me3, H3K9Ac, H3K18Ac and Gcn5 was high in AFSCs derived from anencephalic affected pregnancy and KDM6B levels were low in both MM and anencephalic samples.

FIG. 7 shows bar charts illustrating amniotic fluid and serum levels of BMP4 and Shh. Amniotic fluid (A and C) and serum (B and D) levels of BMP4 and Shh in NTD affected pregnancies, compared to normal. Trends are similar between serum and amniotic fluid. A). Amniotic fluid BMP4 levels were high in MM and low in anencephaly and encephalocele affected pregnancy. BMP4 levels were similar to normal with holoprosencephaly. B). Amniotic fluid Shh was significantly higher with anencephaly. C). Serum BMP4 was high with MM, but non-detectable for anencephaly. D). Serum Shh levels were highest with anencephaly and lowest with MM. Each experiment was done in triplicate.

### DETAILED DESCRIPTION

### Methods of Treating Neural Tube Defects

One aspect of the present provides methods of treating NTDs in a fetus by re-balancing dorsal and ventral neural tube signaling molecules to cause proper neural tube closure and therefore proper differentiation of the neural crest cells. In one embodiment, a composition including noggin or noggin agonist LDN-193189 is administered to the fetus in utero. In another embodiment a composition including sonic hedgehog (Shh) antagonist GDC-0449 (Vismodegib) is administered to the fetus in utero.

It is currently believed that levels of the developmental molecules, bone morphogenetic protein 4 (BMP4) and Shh are critical for proper neural tube development in the fetus. Proper dorso-ventral signaling and patterning leads to appropriate neural tube closure. BMP4 is critical in neural tube dorsalization. Excess BMP4 results in neural tube over dorsalization and ultimately spina bifida. Shh is critical for neural tube ventralization, and must be repressed in the dorsal neural tube for proper neural tube closure. Increased Shh may cause over ventralization and hence lack of neural tube closure. Absence of Shh is associated with midline defect holoprosencephaly, whereas increased Shh is associated with exencephaly and spina bifida.

### Determination of BMP4 and Shh Levels in Maternal Blood and Amniotic Fluid

One embodiment of the present invention provides methods of treating a NTD in a fetus including the in utero administration of noggin or noggin agonist LDN-193189 if the level of BMP4 in the maternal blood or amniotic fluid is above normal. In another embodiment, noggin or noggin agonist LDN-193189 is administered if the level of noggin in the maternal blood or amniotic fluid is below normal.

The inventors have determined that noggin is a folic acid response target and an antagonist of BMP4. BMP4 levels are high and noggin levels low in the serum and amniotic fluid of women with folate responsive NTD affected pregnancy. High BMP4 expression causes over-dorsalization of neural tube and hence no closure.

In one embodiment, noggin is used to counteract the over-dorsalization by excess BMP4. In such conditions, folic acid is not beneficial after a certain gestation period. However, its response target noggin or noggin agonist LDN-193189 can be used to activate the repair process of the open neural tube in utero. This process is termed “Molecular stitching” or stitching the open neural tube using biological or small molecules instead of sutures.

Another aspect of the present invention provides methods of treating a neural tube defect in a fetus including the in utero administration of Shh antagonist GDC-0449 depending on the level of Shh in the maternal blood or amniotic fluid. In cases of folate non-responsive NTD, folic acid supplementation may not rescue NTD. The applicants have determined that folate non-responsive NTDs have elevated sonic hedgehog (Shh) levels as evidenced in folate non responsive NTD animal models. In one embodiment, Shh antagonist GDC-0449 (Vismodegib) is administered to the fetus in utero if the maternal blood or amniotic fluid level of Shh is elevated.

### Determination of Epigenetic Markers in Amniotic Fluid Stem Cells (AFSCs)

Yet another embodiment of the present invention provides methods of treating NTDs including in utero administration of noggin, noggin agonist LDN-193189 or GDC-0449 based on the levels of certain epigenetic markers or histone modifiers in amniotic fluid stem cells (AFSCs), either alone or in combination with levels of at least one of noggin, BMP4 and Shh. In certain embodiments, the marker is a marker for histone modification. In certain other embodiments, the histone modifier is histone acetyltransferase GcnS or histone demethylase KDM6B.

Histones are strongly alkaline proteins found in eukaryotic cell nuclei that package the DNA. Histones can be grouped into five major classes: Hl/H5, H2A, H2B, H3, and H4. These classes are organized into two super-classes: core histones—H2A, H2B, H3 and H4 and linker histones—H1 and H5. Histones undergo posttranslational modifications which alter their interaction with DNA and nuclear proteins. The histones can be covalently modified at several places by, for example methylation, acetylation and phosphorylation. Histone modifications act in diverse biological processes such as gene regulation, DNA repair and chromosome condensation during mitosis. The common nomenclature of histone modifications is the name of the histone followed by the single letter amino acid abbreviation of the amino acid modified and the amino acid position in the protein. Finally, the type of modification is indicated. For example, H3K27me2 denotes the dimethylation of the 27th residue (a lysine) from the N-terminal of the H3 protein.

The epigenetic marker can be a marker for histone methylation or acetylation. In one embodiment, the level of histone methylation or acetylation within the stem cell is determined by a method including by contacting the stem cell with an antibody specific to at least one of histone methylation or acetylation. In yet another embodiment, the level of the histone acetyltransferase Gcn5 within the stem cell is determined by a method including by contacting the stem cell with an antibody specific to Gcn5. In another embodiment, the level of the histone demethylase KDM6B within the stem cell is determined by a method including by contacting the stem cell with an antibody specific to KDM6B. For example, the level of H3K27 methylation within the stem cell is determined by a method including by contacting the stem cell with an antibody specific to at least one of dimethylated histone H3 at K27 and trimethylated histone H3 at K27.

The antibody may be a monoclonal antibody or a polyclonal antibody. In certain embodiments, the antibody is labeled with a detectable probe, for example, a radioactive probe, a fluorescent probe or a chemiluminescent probe. In other embodiments, the antibody is not labeled with a probe. Instead, the presence of the antibody is detected by contacting the antibody with a secondary antibody labeled with such a detectable probe.

In one embodiment, the AFSC is an embryonic stem cell. Maternal amniotic fluid may also contain maternal mesenchymal cells and adult stem cells. In one embodiment, epigenetic or histone modifier markers are determined in embryonic stem cells cultured from amniotic fluid. These cells are allowed to grow into neurospheres, collected, triturated to obtain single cells, and grown further. Neurospheres are established to obviate maternal continuation.

To eliminate maternal cell contamination, amniotic fluid cells are initially cultured in Chang's medium, then moved to Neurobasal medium containing bFGF and EGF, where the cells form Neurospheres. Adult stem cells are difficult to expand in culture and cannot form colonies in Neurobasal medium containing growth factors. The presence of an embryonic stem cell can be demonstrated by the presence of the octamer-binding transcription factor 4 (Oct4) marker. In one embodiment, the presence of Oct4 is determined by a method including contacting the cell with an antibody to Oct4.

In certain embodiments, noggin or noggin agonist LDN-193189 is administered in utero if the maternal blood or amniotic fluid exhibits an elevated BMP4 levels and/or a low noggin level in combination with an above normal or below normal level of at least at least 1, 2, 3, 4, 5, 6, 7 or 8 of H3K4me2, H3K4me3, H3K27me2, H3K27me3, KDM6B, H3K9ac, H3K18ac and GcnS in an AFSC. In one embodiment, noggin or noggin agonist LDN-193189 is administered if the AFSC exhibits an elevated level of at least one of H3K4 methylation and H3K27 methylation or a decreased level of at least one of H3K9 acetylation, H3K18 acetylation and GcnS.

In another embodiment, Shh antagonist GDC-0449 is administered to the fetus in utero if the maternal blood or amniotic fluid exhibits an elevated Shh level in combination with an above normal or below normal level of at least at least 1, 2, 3, 4, 5, 6, 7 or 8 of H3K4me2, H3K4me3, H3K27me2, H3K27me3, KDM6B, H3K9ac, H3K18ac and GcnS in an AFSC. In one embodiment, GDC-0449 is administrated if the AFSC exhibits an elevated level of at least one of 1-13K9 acetylation, H3K18 acetylation and GcnS.

### Noggin, BMP4 and Shh as Biomarkers for Neural Tube Defects

Another aspect of the present invention provides methods of determining the presence of a NTD based on the level of a biomarker in the maternal blood or amniotic fluid. In one embodiment, the presence of a folate responsive NTD is indicated by a depressed level of noggin and/or an elevated level of BMP4 in the material blood. In another embodiment, the presence of a folate non-responsive NTD is indicated by an elevated Shh level in the material blood.

### Epigenetic Markers and Histone Modifiers in AFSCs as Biomarkers for Neural Tube Defects

Another aspect of the present invention provides methods of determining the presence of a NTD based on the level of an epigenetic marker or histone modifier in an AFSC, either alone or in combination with maternal blood or amniotic fluid levels of noggin, BMP4 or Shh. In one embodiment, the presence of a NTD is indicated by an above normal or below normal level of at least 1, 2, 3, 4, 5, 6, 7 or 8 of H3K4me2, H3K4me3, H3K27me2, H3K27me3, KDM6B, H3K9ac, H3K18ac and GcnS in an AFSC. In another embodiment, alternations in H3K27me2/3 levels are associated with MM. In yet another embodiment, alternations of H3K27me3, H3K9/18ac and GcnS levels are associated with anencephaly. In another embodiment, the presence of MM is indicated by increased levels of H3K4me2, H3K4me3, H3K27me2 and H3K27me3 and a decreased level of KDM6B.

### Kits, Reagents and Compositions for Detecting and Treating Neural Tube Defects

The present invention also provides for kits and reagents for detecting NTDs. In one embodiment, the kit contains at least a reagent including an antibody specific to at least one of H3K4me2, H3K4me3, H3K27me2, H3K27me3, KDM6B, H3K9ac, H3K18ac and GcnS in combination with a buffer in a package or container. In another embodiment, the kit contains at least a reagent comprising an antibody specific to at least one of noggin, BMP4 and Shh. The antibody may be a monoclonal antibody or a polyclonal antibody. In certain embodiments, the antibody is labeled with a detectable probe, for example, a radioactive probe, a fluorescent probe or a chemiluminescent probe. In other embodiments, the antibody is not labeled with a probe. Instead, the kit further includes a secondary antibody labeled with such a detectable probe. In these embodiments and in the embodiments described below the buffer can be in a liquid, frozen or a freeze dried form.

In certain embodiments, the kit also includes a reagent including an embryonic stem cell marker, such as Oct4, in combination with a buffer in a package or container. In another embodiment, the kit also includes a reagent including a nuclear stain, such as DAPI, in combination with a buffer in a package or container.

In other embodiments, the kit includes one or more wash buffers, (for example, Phosphate buffered saline) and/or blocking buffers (for example, 5% Normal Donkey Serum/0.01% Triton X-100/0.01% sodium azide in PBS) in packages or containers. In yet other embodiments, the kits may include a signal generation reagent for development of a detectable signal from the signaling moiety. The kits may also include one or more sample collection devices, for example a syringe or needle suitable for performing a lumbar puncture. In other embodiments, the kits also include positive and/or negative control samples in suitable packages or containers.

When a kit is supplied, the different components may be packaged in separate containers and admixed immediately before use. Such packaging of the components separately may permit long-term storage without losing the active components' functions. Kits may also be supplied with instructional materials. Instructions may be printed on paper or other substrate, and/or may be supplied as an electronic-readable medium.

Another aspect of the present invention provides compositions and kits for treatment of NTDs. One embodiment provides a composition including of therapeutically effective amount of noggin, LDN-193189 or GDC-0449 and a pharmaceutically acceptable carrier. “Pharmacologically acceptable” refers to ligands, materials, compositions, and/or dosage forms which are, within the scope of sound medical judgment, suitable for use in contact with the tissues of humans without excessive toxicity, irritation, allergic response, or other problem or complication, commensurate with a reasonable benefit/risk ratio. A “therapeutically effective amount” of noggin, LDN-193189 or GDC-0449, with respect to use in treatment of a NTD, refers to an amount which, when administered as part of a desired dosage alleviates a symptom of a NTD according to clinically acceptable standards. In one embodiment, the composition is supplied in a form suitable for in utero delivery, for example, the composition may be supplied in a form suitable for in utero injection for delivery to the neural tube of a fetus.

Another aspect of the present invention provides a kit for the treatment of a NTD. In one embodiment, the kit includes a reagent including a therapeutically effective amount of noggin, LDN-193189 or GDC-0449 and a pharmaceutically acceptable carrier. The kit may also include a syringe of other device suitable for delivering the composition to the neural tube of a fetus.

### EXAMPLES

### Example 1

### Collection of Human Amniotic Fluid and Serum Samples

Amniotic fluid samples were collected from six pregnant women through amniocentesis, between 16 and 18 weeks of gestation. Two samples were from women exhibiting normal pregnancies, and one each showing myelomeningocele (MM), anencephaly, encephalocele and holoprosencephaly. All women who donated amniotic fluid and blood samples signed informed consent forms (IRB approval #STU00012913; Northwestern University Feinberg School of Medicine, Chicago, Ill.).

### Example 2

### Primary Culture of Human Amniotic Fluid Cells and Isolation of Amniotic Fluid Stem Cells (AFSCs)

Amniotic fluid samples (5 ml) were filtered using 100-μm filters, and centrifuged at 400 g (4° C.) for 10 min. Supernatant was stored for later use, precipitates were seeded in 75-mm tissue culture dishes with hAFC medium (a-MEM medium (Gibco, Invitrogen) containing 15% ES-grade FBS, 1% glutamine and 1% penicillin/streptomycin (Gibco, Invitrogen) supplemented with 18% Chang B and 2% Chang C (Irvine Scientific)), and incubated at 37° C. with 5% humidified CO2 . Non-adhering cells were removed on the fifth day after seeding. New media was added to adherent cells, which were than maintained until 65-70% confluence. Cells obtained from amniotic fluid associated with encephalocele and holoprosencephaly affected pregnancies did not survive in culture. Amniotic fluid received from these samples was slightly turbid and tinted red, it is possible that these samples were left at RT for an extended period of time, resulting in cells lysis.

To isolate AFSCs, cells from the primary cell culture were grown in Neurobasal plus medium supplemented with bFGF (20 nanogram/ml) and EGF (20 ng/ml, in flasks precoated with Po1yHEMA. The cells were grown for 7 days during which they formed floating neurospheres (colony forming units). For differentiation studies, the neurospheres were grown in Neurobasal medium devoid of growth factors on 8 chambered slide coated with laminin, and cell allowed to differentiate for 7 days.

### Example 3

### Immunostaining

On day 7 individual colonies >50 μm, were selected and plated in the absence of growth factors in 8 well chamber slides pre-coated with laminin (Sigma L2020). Adherent neurospheres were fixed in 4% paraformaldehyde in PBS. CD133 (Abeam ab19898-100), Sox2 (Chemicon AB5603), Oct4 (Santa Cruz Biotechnology sc-8629), alkaline phosphatase (Millipore SCR004); and the neural progenitor marker Nestin (Millipore MAB353) were used to confirm stem cell characteristics. Immunostaining was done as described by Ichi et al. (Ichi, et al. J. Biol. Chem 285: 36922-36932 2010). Antibodies for epigenetic markers included: H3K4me2 (ab8580), H3K4me3 (ab7766), H3K27me2 (ab24684) and KDM6B (rabbit polyclonalab38113), from Abeam; H3K27me3 (rabbit polyclonal #07-449) from Upstate; H3K9ac (rabbit poly-CS#9671S) and H3K18ac (rabbit poly-CS#9675S) from Cell Signaling); and GcnS (goat polyclonal—sc6303) from Santa Cruz. Prior to treatment with primary antibodies cells were blocked with 10% normal donkey serum/0.1% Triton X-100/0.01% Sodium azide in PBS. Secondary antibodies included: Cy3-conjugated donkey anti-rabbit & anti-mouse IgG (Jackson ImmunoResearch) and Alexa Fluor 488-labeled donkey anti-rabbit & goat IgG (Invitrogen). Nuclear staining was done with DAPI. For densitometry all immunostained sections were examined at identical settings (excitation/offset etc.), on a Leica microscope.

### Example 4

### BMP4 and Shh Enzyme Linked Immuno-Sorbent Assays (ELISA)

BMP4 and Shh levels from amniotic fluid and serum samples were diluted and measured using an ELISA kit (Abeam, Cambridge, Mass. 02139) and antibodies against human BMP4 (ab99982) and human Shh (ab100639).

### Example 5

### Epigenetic Markers in AFSCs of MM and Anencephaly Affected Pregnancies

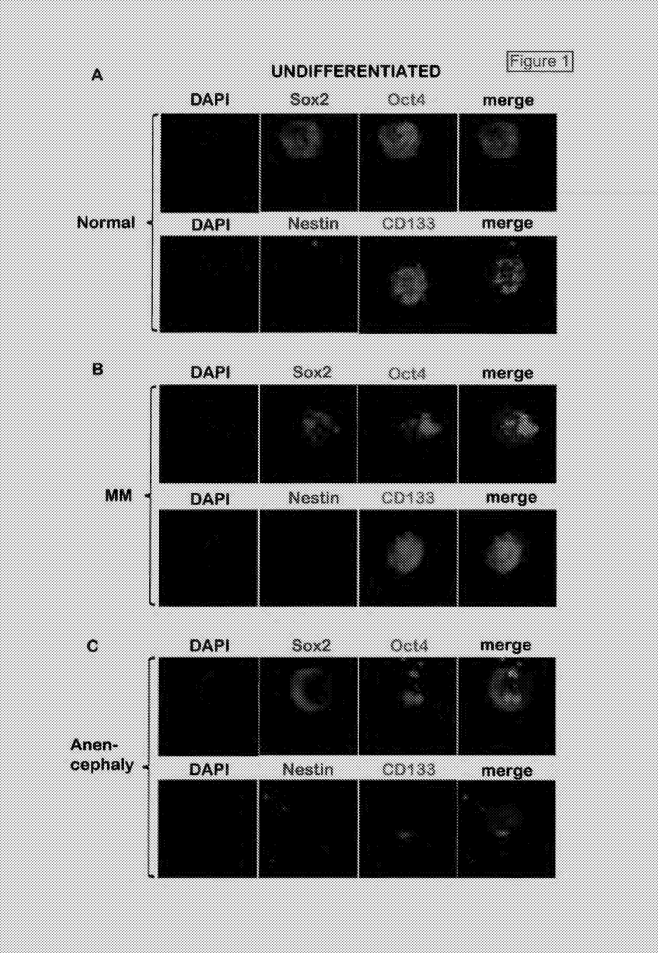

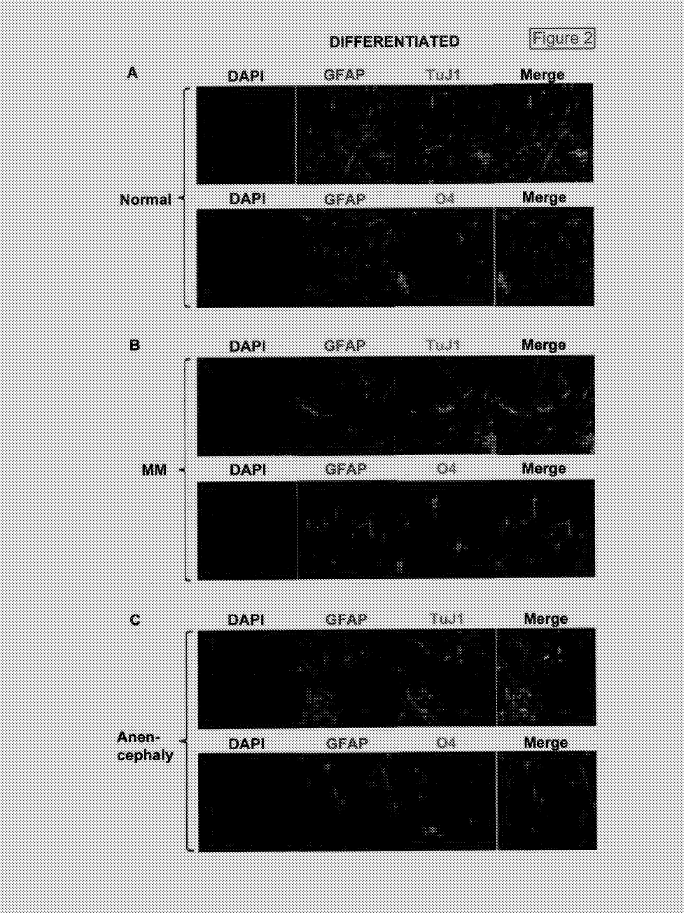

Epigenetic markers for chromatin inactivation, i.e. H3K27me3, and activation i.e. H3K4me2/3 and histone acetylation have been associated with key developmental gene promoters. To ascertain whether methylation and acetylation profiles are altered in pregnancies complicated by NTD, AFSCs were grown in culture and immunostained with antibodies against H3K4me2, H3K4me3, H3K27me2, H3K27me3, H3K9ac, H3K18ac KDM6B and Gcn5. AFSCs were of embryonic origin as opposed to mesenchymal maternal cells. These cells survived and formed neurospheres in culture, whereas maternal mesenchymal cells do not form neurosphere colonies in Neurobasal Plus (growth factor supplemented) medium. Stem cells obtained from normal, MM, and anencephalic pregnancies were positive for the stem cell markers Oct4, CD133 and Sox2; and for the neural progenitor marker, nestin (FIG. 1). Finally neurosphere colonies differentiated into different lineages, i.e. neurons, astrocytes and oligodendrocytes, as evidenced by TuJ1, GFAP and O4 staining, respectively (FIG. 2).

### Example 6

Determination of H3K4me2/3, H3K27me2/3 and KDM6B Levels

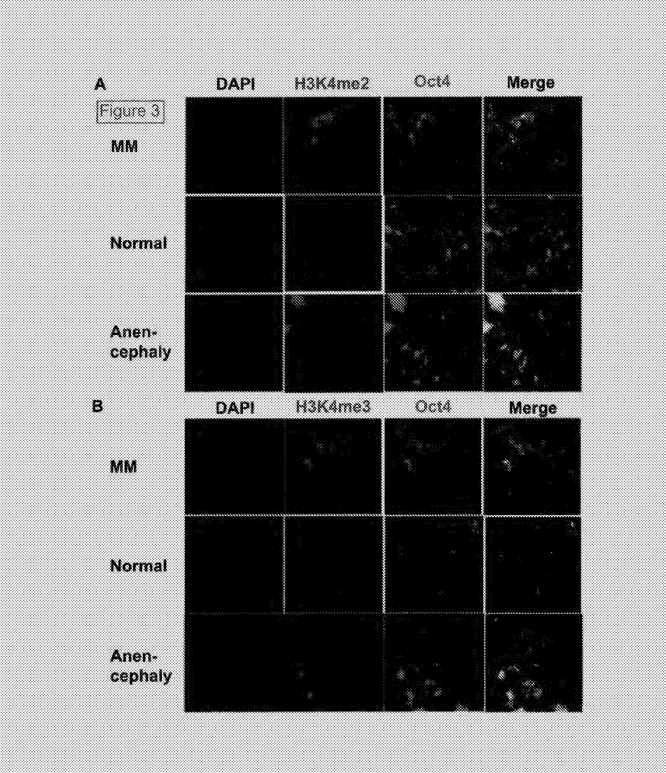

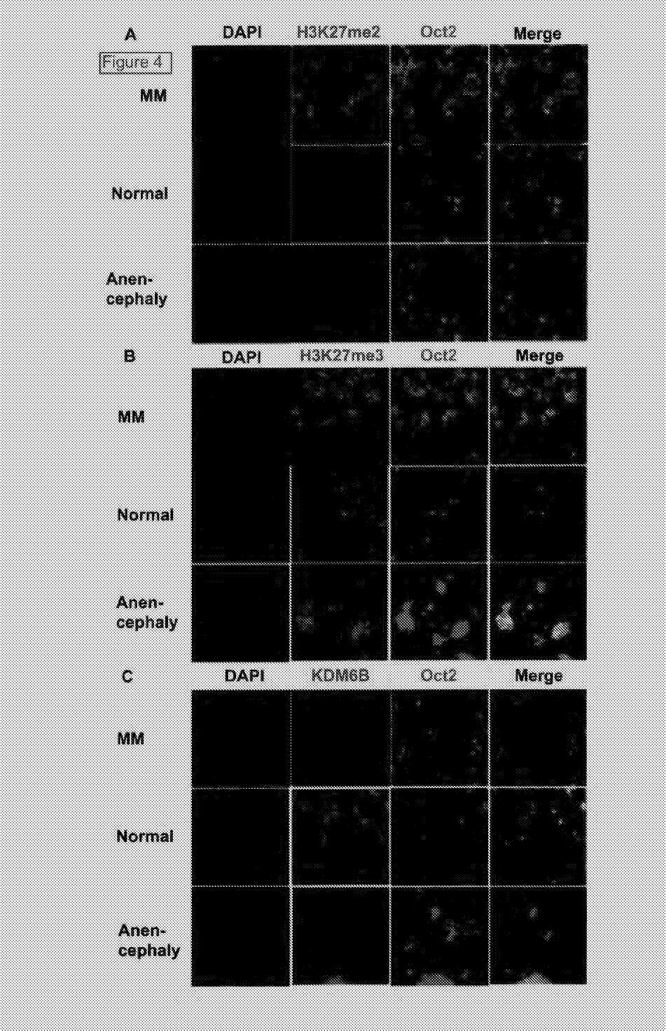

To determine if H3 methylation patterns change in NTD affected pregnancy, immunostaining was performed on cultured AFSCs from normal, MM and anencephaly affected pregnancies for H3K4me2, H3K4me3, H3K27me2, H3K27me3 and KDM6B. H3K4me2 and H3K4me3 (FIGS. 3A and B) levels increased in MM AFSCs compared to normal controls. Levels of these markers did not change with anencephaly. H3K27me2 and H3K27me3 were high in AFSCs cultured from MM affected pregnancy (FIGS. 4A and B), KDM6B levels decreased. This corroborated with animal data from Ichi et al. (Ichi, et al. J. Biol. Chem 285: 36922-36932 2010), showing increased H3K27 methylation in neural crest stem cells from Sp-/-embryos, compared to WT. In anencephaly H3K27me3 levels increased, and KDM6B levels decreased (FIG. 4C).

### Example 7

### Determination of H3K9Ac and H3K18Ac and GcnS Levels

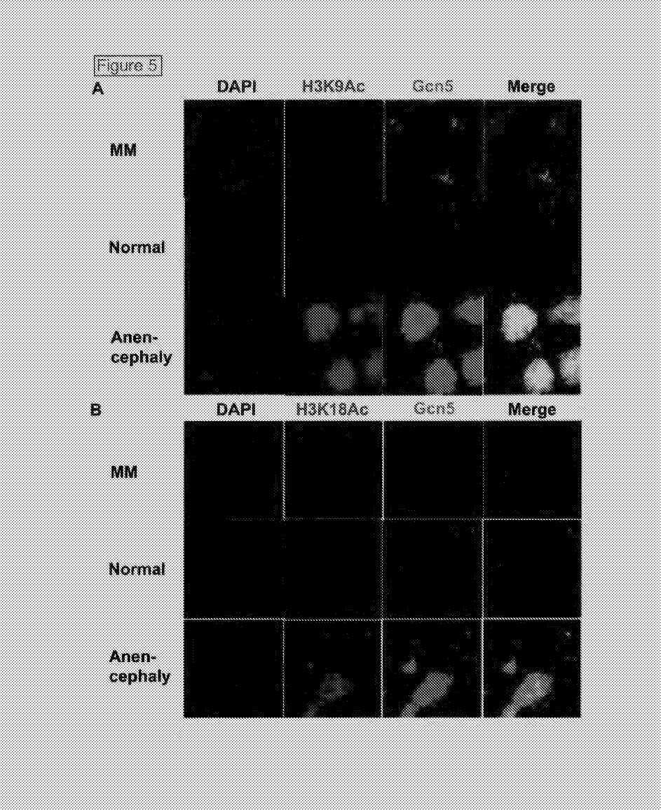

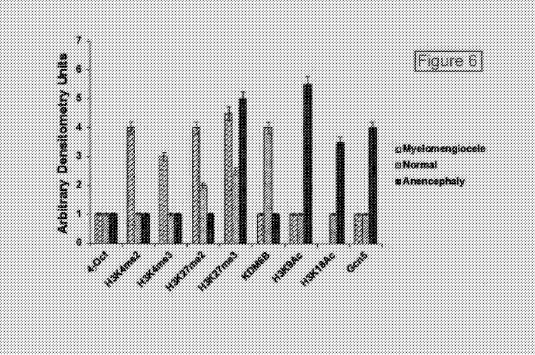

H3K9ac and H3K18ac are associated with active chromatin34-36. In AFSC cultured from the MM associated pregnancy, H3K9ac and H3K18Ac levels were comparable to normal. In AFSC cells cultured from the anencephaly associated pregnancy, H3K9Ac and H3K18Ac increased (FIG. 5B). Gcn5, an H3K9 and H3K18 acetyltransferase, increased with anencephaly, but not with MM. In sum, AFSCs cultured from anencephaly affected pregnancy express higher levels of H3K9Ac, H3K18Ac and GcnS compared to cells from MM and normal pregnancy. Overall, the data (densitometry data summarized in FIG. 6) for epigenetic markers in AFSCs suggest that (i) alterations in H3K27me2 and H3K27me3 are associated with MM affected pregnancy; (ii) whereas alterations in H3K27me3, H3K9/18ac and Gcn5 are associated with anencephaly affected pregnancy.

### Example 8

### Determination of BMP4 and Shh Levels in Serum and Amniotic Fluid

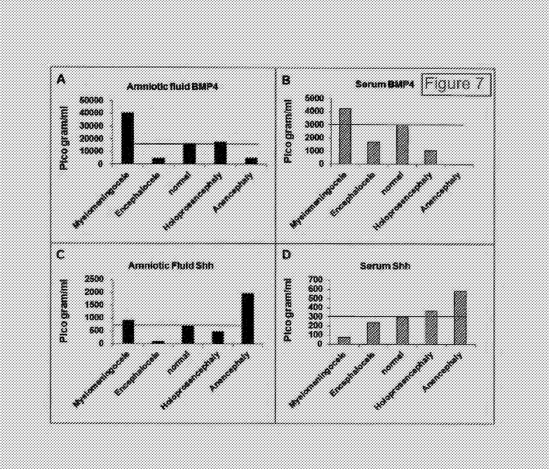

Increased H3K27 methylation has been associated with decreased expression of noggin, a BMP4 antagonist, thereby resulting in increased BMP activity in developing spinal cord. Since we observed increased H3K27 methylation in AFSC, it was hypothesized that BMP4 levels would be increased in amniotic fluid and serum from NTD affected pregnancies. BMP4 levels were examined using ELISA. Compared to normal, BMP4 levels were high in amniotic fluid and serum taken from the patient with a fetus presenting with MM. BMP4 levels were low in amniotic fluid and serum for anencephaly and encephalocele affected pregnancies; serum BMP4 levels were also low with holoprosencephaly (FIGS. 7A and B). Shh levels in amniotic fluid and serum were high from the patient whose fetus presented with anencephaly and low from patients whose fetuses presented with MM, encephalocele and holoprosencephaly (FIGS. 7C and D).

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method of treating a neural tube defect in a fetus, the method comprising administrating a composition comprising noggin or LDN-193189 in utero.

2. The method of claim 1, further comprising measuring the amount of bone morphogenetic protein 4 (BMP4) in a sample taken from an individual carrying the fetus, wherein the composition is administrated if the sample contains an elevated amount of BMP4.

3. The method of claim 2, wherein the sample is a blood sample or an amniotic fluid sample.

4. The method of claim 1, wherein the neural tube defect is an open neural tube and wherein the composition is applied locally to the neural tube.

5. The method of claim 1, wherein the composition is injected in-utero.

6. The method of claim 1, wherein the neural tube defect is a folate responsive neural tube defect.

7. The method of claim 1, further comprising:
isolating an embryonic stem cell from amniotic fluid an individual carrying the fetus; and
determining the level of at least one of H3K4 methylation, H3K27 methylation, H3K9 acetylation, H3K18 acetylation, and GcnS within the stem cell,
wherein the composition is administrated if the stem cell exhibits an elevated level of at least one of H3K4 methylation and H3K27 methylation or a decreased level of at least one of H3K9 acetylation, H3K18 acetylation and Gcn5.

8. The method of claim 7, wherein the determining of the level of H3K27 methylation in the stem cell comprises contacting the stem cell with antibody to at least one of dimethylated histone H3 at K27 and trimethylated histone H3 at K27.

9. The method of claim 7, further comprising determining the presence or absence in the stem cell of octamer-binding transcription factor 4 (Oct4), wherein the determining the presence or absence of Oct4 comprises contacting the stem cell with an antibody to Oct4.

10. The method of claim 9, wherein the composition is administrated if Oct4 is present in the stem cell and the stem cell exhibits an elevated level of at least one of H3K4 methylation and H3K27 methylation, or a decreased level of at least one of H3K9 acetylation, H3K18 acetylation, KDM6B and Gcn5

11. A method of treating a neural tube defect in a fetus, the method comprising administrating a composition comprising GDC-0449 in utero.

12. The method of claim 11, further comprising measuring the amount of sonic hedgehog (Shh) in a sample taken from an individual carrying the fetus, wherein the composition is administrated if the sample contains an elevated amount of sonic hedgehog (Shh).

13. The method of claim 12, wherein the sample is a blood sample or an amniotic fluid sample.

14. The method of claim 11, wherein the neural tube defect is an open neural tube and wherein the composition is applied locally to the neural tube.

15. The method of claim 11, wherein the neural tube defect is a folate non-responsive neural tube defect.

16. The method of claim 11, further comprising:
isolating an embryonic stem cell from amniotic fluid an individual carrying the fetus;
determining the level of at least one of H3K9 acetylation, H3K18 acetylation, and Gcn5 within the stem cell,
wherein the composition is administrated if the stem cell exhibits an elevated level of at least one of H3K9 acetylation, H3K18 acetylation and Gcn5.

17. The method of claim 16, wherein the determining of the level of H3K9 acetylation in the stem cell comprises contacting the stem cell with antibody to H3K9Ac and wherein the determining of the level of H3K18 acetylation in the stem cell comprises contacting the stem cell with antibody to H3K18Ac.

18. The method of claim 16, further comprising determining the presence or absence in the stem cell of octamer-binding transcription factor 4 (Oct4), wherein the determining of the Oct4 comprises contacting the stem cell with an antibody to Oct4.

19. The method of claim 18, wherein the composition is administrated if Oct4 is present in the stem cell and the stem cell exhibits an elevated level of at least one of H3K9 acetylation, H3K18 acetylation and Gcn5.
